(19) United States
(12) Patent Application Publication
(10) Pub. No.: US 2024/0233913 A9
(43) Pub. Date: Jul. 11, 2024

(54) USER ANALYSIS AND PREDICTIVE TECHNIQUES FOR DIGITAL THERAPEUTIC SYSTEMS

(71) Applicant: CURIO DIGITAL THERAPEUTICS, INC., Princeton, NJ (US)

(72) Inventors: Sidhartha SHANKAR, Skillman, NJ (US); Shailja DIXIT, Skillman, NJ (US)

(21) Appl. No.: 18/491,521

(22) Filed: Oct. 20, 2023

Related U.S. Application Data
(60) Provisional application No. 63/380,312, filed on Oct. 20, 2022.

(51) Int. Cl. G16H 20/70; A61B 5/00; A61B 5/16

(52) U.S. Cl. CPC G16H 20/70; A61B 5/165; A61B 5/4803; A61B 5/7275

(57) ABSTRACT

Systems and methods for analyzing users interacting with or utilizing a digital therapeutic system are disclosed. The systems and methods include receiving audio and video of the user in connection with the user interacting with or receiving digital therapy content, determining speech data or other user data from the audio and video, determining a mental state for the user based on the determined data, and taking an action based on the determined mental state, such as providing the determined mental state to at least one of the user or a healthcare provider associated with the user.

### PRIORITY

The present application claims priority to U.S. Provisional Patent Application No. 63/380,312, titled USER ANALYSIS AND PREDICTIVE TECHNIQUES FOR DIGITAL THERAPEUTIC SYSTEMS, filed Oct. 20, 2022, which is hereby incorporated by reference herein in its entirety.

### BACKGROUND

A state of mind indicates a mood or mental status, which includes a diverse class such as emotion, desire, and pain experience. The state of mind of a person is detected by analyzing some of the vital signs of the body that are indicators of internal body health such as body temperature, heart rate, blood pressure, and/or rate of breathing. Variations in these vital signs indicate changes in human health physically and mentally and through a psychological evaluation in which a mental health expert communicates with the patient to know about the patient's thoughts, behavior patterns, and/or the like. The measurements of vital signs and results of the psychological evaluation are analyzed to detect the state of mind of a person.

Currently, the patient has to visit a clinic or lab where the patient's vital signs are measured and a psychological evaluation is performed physically by a mental health expert. However, there are some scenarios where a patient is not able to physically visit or meet with the mental health expert. In such a scenario, the patient takes an online mode consultation in which the mental health expert conducts the psychological evaluation. However, the mental health expert is unable to detect the real time state of mind of the patient in this scenario. For instance, if a patient is suffering from depression or postpartum depression, the patient may convey to the doctor that he/she is feeling good or fine, but is really internally suffering from depression. In that case, the health expert is not able to detect the real time state of mind accurately, which can cause serious problems such as an increase in the chance of risky behaviors and/or problems at work and/or relationships. If left untreated, a mild case of depression may transform into a serious illness, which makes it difficult to overcome.

To resolve this issue, several systems and devices were introduced for detecting the state of mind of a person. The patient is asked to respond to a set of questions honestly. Those answers are matched with data stored in a database, and the state of mind is predicted. However, these systems and devices are not able to predict an accurate state of mind of the patient because the patient may provide false answers. Some advanced systems of detecting the state of mind of a person are also available in the market that comprises sweat sensors, ECG sensors and the like, but these advanced systems are not user friendly and require health care experts or similar professionals to set up and operate these systems.

To overcome the aforementioned drawbacks, machine learning techniques were used to detect and monitor the state of mind through facial expression or by analyzing the speech, emotions, and actions of the patient. However, accurate results from such techniques were still not achieved. The machine learning-based systems take an input image or video and process the input to predict the patient's state of mind. However, there are cases where the patient may seem normal physically, but internally feels depressed. Hence, the currently available systems, methods, or devices are not highly reliable.

Mood detection systems are also used to detect the state of mind of a person, but an incorrect emotion prediction can lead to false detection of the patient's state of mind because currently available mood detectors are only able to detect basic aspects of the patient's mood and fail to detect the complex mood of a person. Similarly, stress detectors are also available in the market that are integrated within fitness bands, which processes the patient's heart rate and/or breathing rate to predict the amount of stress. However, it is impossible to wear fitness bands throughout the day. Moreover, radiation from these devices may cause serious illness to the patient who is suffering from any type of depression or any type of neurological disorder.

In particular, pregnant women experience anxiety during the postpartum period and, even at subclinical levels, this has highly detrimental and long-term effects on mothers and their infants. Despite ˜20% prevalence of postpartum depression (PPD), it is infrequently diagnosed and treated, often because of stigma, lack of awareness, and insufficient investment in the mental health infrastructure. The ready availability of large health claims databases affords an opportunity and means to develop AI tools for identifying and quantifying the disease risk at an early stage, thereby allowing earlier intervention and better outcomes with a significant reduction in associated costs.

Therefore, there is a need for systems that are able to assess individuals' states of mind and their risk for PPD in order to provide appropriate support to women, whether currently pregnant and post-pregnancy, that they are lacking under the current healthcare regime.

### SUMMARY

The present disclosure is directed to systems and methods for analyzing and making predictions for users based on their interactions with a digital therapeutic system. In some embodiments, the systems and methods could be configured to predict users' states of mind based on their interactions with the digital therapeutic system and/or the content provided thereby. In some embodiments, the systems and methods could be configured to identify potentially at-risk individuals. By analyzing users in these manners, digital therapeutic systems can tailor content provided to the users, provide notifications to alert users and/or healthcare providers as to when the user may need additional support, and take other beneficial actions.

In one embodiment, the present disclosure is directed to a computer system for providing digital therapy content to a user via a user device, the user device comprising a camera and a microphone for recording audio and video of the user, the computer system comprising: a processor; and a memory coupled to the processor, the memory storing instructions that, when executed by the processor, cause the computer system to: provide the digital therapy content to the user device, receive the audio and the video of the user from the user device in connection with the digital therapy content, determine a speech-based biomarker associated with the user from the audio, determine a visual-based biomarker associated with the user from the video via remote photoplethysmography, determine a mental state for the user based on at least one of the determined speech-based biomarker or the determined visual-based biomarker of the user, and provide the determined mental state to at least one of the user or a healthcare provider associated with the user.

In one embodiment, the present disclosure is directed to a computer-implemented method for providing digital therapy content to a user via a user device, the user device comprising a camera and a microphone for recording audio and video of the user, the method comprising: providing, by a computer system, the digital therapy content to the user device; receiving, by the computer system, the audio and the video of the user from the user device in connection with the digital therapy content; determining, by the computer system, a speech-based biomarker associated with the user from the audio; determining, by the computer system, a visual-based biomarker associated with the user from the video via remote photoplethysmography; deter-mining, by the computer system, a mental state for the user based on at least one of the determined speech-based biomarker or the determined visual-based biomarker of the user; and providing, by the computer system, the determined mental state to at least one of the user or a healthcare provider associated with the user.

In some embodiments, the determined mental state is provided via a user interface of a digital therapy app executed by the user device, wherein the digital therapy app is communicably coupled to the computer system.

In some embodiments, the digital therapy content is configured for treatment of postpartum depression.

In some embodiments, the memory further stores a machine learning model trained to identify a distress condition based on audio input data, wherein the speech marker is determined based on the machine learning model.

In some embodiments, the mental state is determined based on both the determined speech biomarker and the determined visual biomarker.

In some embodiments, the mental state comprises at least one of anxiety, depression, or post-traumatic stress disorder.

In some embodiments, the method further comprises adjusting, by the computer system, the digital therapy content provided to the user based on the determined state of mind.

### BRIEF DESCRIPTION OF THE DRAWINGS

FIG. 1 shows a diagram of a digital therapeutic system and systems for interacting therewith in accordance with an embodiment of the present disclosure.

FIG. 2A shows a flow diagram of a first process for analyzing a user's state of mind. in accordance with an embodiment of the present disclosure.

FIG. 2B shows a flow diagram of a second process for analyzing a user's state of mind in accordance with an embodiment of the present disclosure.

FIG. 2C shows a flow diagram of a third process for analyzing a user's state of mind in accordance with an embodiment of the present disclosure.

FIG. 3 shows a diagram of various ML-based approaches for analyzing audio data in accordance with an embodiment of the present disclosure.

FIG. 4 shows a diagram of a process for analyzing a user's heartbeat from a video relative to baseline data in accordance with an embodiment of the present disclosure.

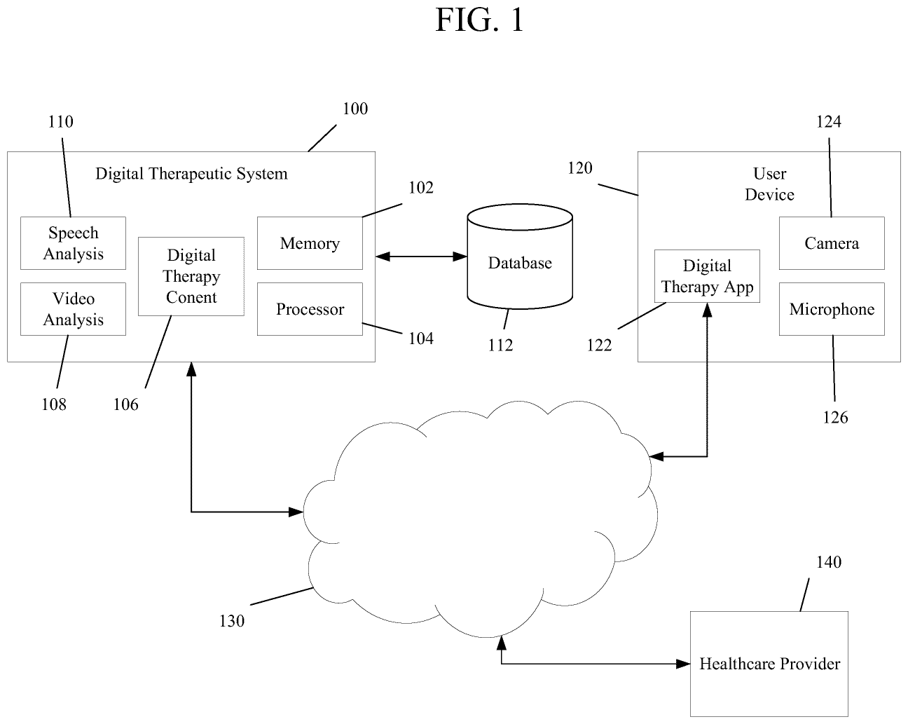

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “pharmaceutical” is a reference to one or more pharmaceuticals and equivalents thereof known to those skilled in the art, and so forth.

As used herein, the term “about” means plus or minus 10% of the numerical value of the number with which it is being used. Therefore, about 50 days means in the range of 45 days to 55 days.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of”

As used herein, the term “module” refers to hardware, firmware, software, or any combination thereof that is operable to provide the specified functionality.

### Digital Therapeutic System

The present application is generally directed to the provision of digital therapeutic services to users. In one embodiment, a digital therapeutic system 100 may be accessed via a user device 120 through a network 130 (e.g., the Internet or another telecommunication network). The digital therapeutic system 100 may provide digital therapy content 106 to a user through the device 120. The digital therapeutic system 100 may include a computer system, such as a server or server system, that is configured to provide the digital therapy content 106 to a user through the user device 120. The digital therapeutic system 100 may further include a memory 102 and a processor 104 that is adapted to execute instructions stored in the memory 102 to provide the digital therapy content 106 to the user device 120 and perform other tasks described herein. The user device 120 may include a mobile device (e.g., a smartphone), a tablet, a laptop, a desktop computer, or any other device that is able to access and/or display the digital therapy content 106. In one embodiment, the user may download a smartphone app 122 on the user device 120 through which the digital therapeutic system 100 can be accessed to provide the digital therapy content 106 thereto. In other embodiments, the digital therapeutic system 100 may be accessed via, for example, a website, a web application, or as a software as a service (SaaS) model. The digital therapy app 122 may provide a user interface through which the user can access, view, and/or interact with the digital therapy content 106 provided via the digital therapeutic system 100.

As one potential implementation, the digital therapeutic system 100 may be configured to provide digital therapeutic services associated with or otherwise related to pregnancy. In one embodiment, the digital therapy content 106 may be designed to manage symptoms of depression and anxiety during pregnancy or after delivery. In particular, the digital therapy content 106 may be designed for developing skills to help manage symptoms of anxiety and depression, promoting social support and relationship quality, and encouraging help-seeking behavior in users. In one embodiment, the digital therapy content 106 provided via the digital therapy app 122 may encourage users to upload audio and/or video content, which may in turn be received by the digital therapeutic system 100. For example, the digital therapy content 106 may request that users upload video (e.g., video journal entries) and/or audio of themselves. The digital therapy content 106 may request that users upload the video and/or audio on a periodic (e.g., daily), a nonperiodic basis, or in response to various user inputs or other parameters. To facilitate the recording of the video and/or audio content, the user device 120 may include a camera 124, a microphone 126, and/or other recording devices. The uploaded user video and/or audio may be stored in, e.g., a database 112 associated with the digital therapeutic system 100. In some embodiments, which are described in greater detail below, the digital therapeutic system 100 may be configured to analyze the user video and/or audio (as well as other user data) for predictive analytics in order to tailor the digital therapy content 106 delivered to the user, notify the user as to any detected trends, and/or notify healthcare professionals accordingly. In particular, the digital therapeutic system 100 may include a video analysis module 108, a speech analysis module 110, or a combination thereof. The video analysis module 108 and/or speech analysis module 110 may be embodied as instructions stored in the memory 102 that are executable by the processor 104 to perform the described tasks. The video analysis module 108 may be configured to analyze the video content uploaded by the user for predictive analytics, such as is described in greater detail below. Likewise, the speech analysis module 110 may be configured to analyze the audio data (e.g., recorded speech) uploaded by the user for predictive analytics, such as is described below and in U.S. patent application Ser. No. 17/725,145, titled A SYSTEM FOR REAL TIME DETECTION AND ANALYSIS OF SPECIFIC SPEECH BIOMARKERS, filed Apr. 20, 2022, which is hereby incorporated by reference herein in its entirety.

In one embodiment, the digital therapeutic system 100 may further be communicably connected to a healthcare provider 140 associated with the user. For example, the digital therapeutic system 100 may be configured to upload data to an electronic medical record (EMR) associated with the user, send a message (e.g., email) to the user's healthcare professional, or update a user profile provided by the digital therapeutic system 100 that is accessible by the healthcare provider 140. In one embodiment, the digital therapeutic system 100 may only notify the healthcare provider 140 in response to appropriate permissions granted by the user.

The digital therapy content 106 may be designed to provide personalized self-help tools for women, such as women attempting to manage symptoms of depression and anxiety during pregnancy or after delivery. The digital therapy content 106 may guide expecting and new mothers through their journey, easing the transition to parenthood and providing helpful tips, self-guided strategies and reminders along the way. The digital therapy content 106 may be designed to be completed over a particular time period (e.g., 8 weeks). The digital therapy content 106 may include a series of modules focused on developing skills to help manage symptoms of anxiety and depression, promoting social support and relationship quality, and encouraging help-seeking behavior. Digital tools, such as provided by the digital therapy content 106, are useful in delivering self-guided personal development and treatment strategies due to their flexibility, privacy, personalization, and ease-of-use. Further, the digital therapy content 106 may include interactive exercises that provide personalized feedback to support learning and built-in trackers make it easy for users to track their progress through the digital therapy content 106.

In some embodiments, the digital therapy content 106 may be designed to provide cognitive behavioral therapy (CBT) to users. Accordingly, the digital therapy content 106 may include one or more modules providing CBT content that users can interact with or view to receive CBT. The digital therapy content 106 may, for example, by developed by or in concert with clinical psychologists and other experts to encourage development of skills to help manage the symptoms of depression and anxiety using CBT-based principles. The MamaLift program addresses the minimization of risk factors for postpartum depression, including lack of social support, along with the promotion of psychological processes and self-regulatory skills such as emotion regulation, psychological flexibility and self-compassion.

### State of Mind Analysis

As described above, users interact with the digital therapeutic system 100 to, for example, receive digital therapy content 106 therefrom. As part of the interactions with the digital therapeutic system 100, users may upload video and/or audio recordings of themselves on a regular (e.g., daily) basis. Advantageously, the digital therapeutic system 100 may leverage the uploaded video and/or audio generated from a user's interactions with the system 100 to monitor the user's state of mind. In particular, the digital therapeutic system 100 may identify one or more biomarkers associated with the user based on the audio and/or video data and, accordingly, determine the user's state of mind using machine learning and algorithmic techniques. In some embodiments, the digital therapeutic system 100 may also take a variety of different actions based on the user's detected state of mind, such as adjusting the digital therapy content provided to the user or providing notifications to the user and/or a healthcare provider 140.

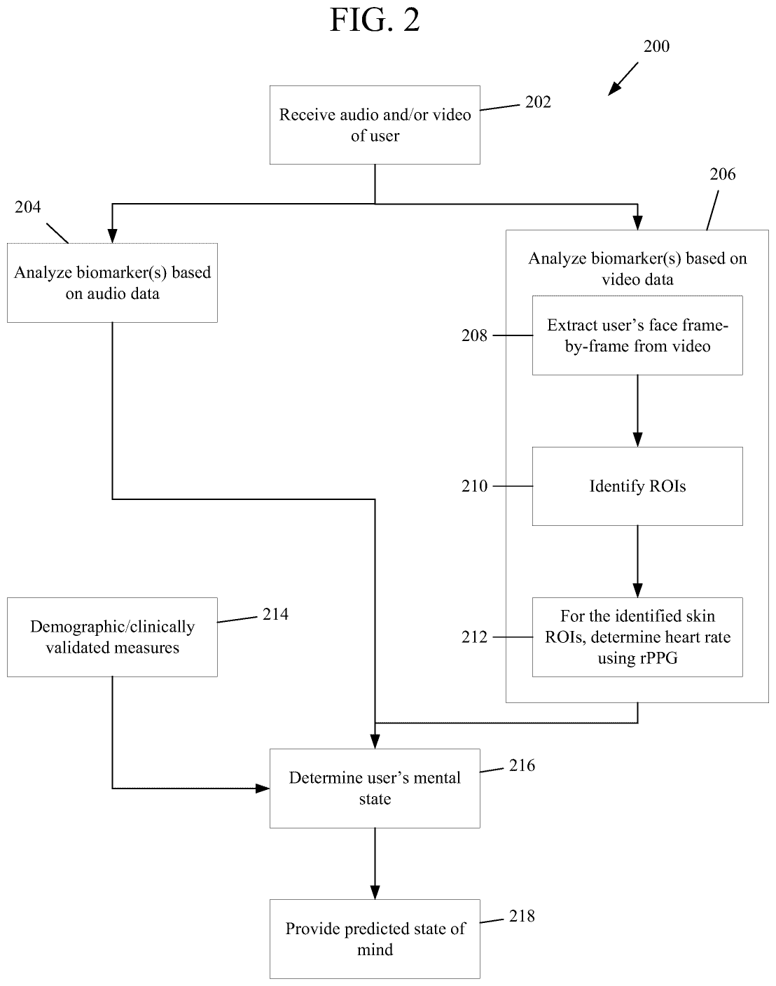

One embodiment of a process 200 for analyzing users' states of mind is shown in FIG. 2. In one embodiment, the process 200 may be embodied as instructions stored in a memory (e.g., the memory 102) that, when executed by a processor (e.g., the processor 104), cause the digital therapeutic system 100 to perform the process 200. In various embodiments, the process 200 may be embodied as software, hardware, firmware, and various combinations thereof. In various embodiments, the process 200 may be executed by and/or between a variety of different devices or systems. For example, various combinations of steps of the process 200 may be executed by the digital therapeutic system 100, the network 130, and/or the user device 120 (e.g., computer, laptop, or smartphone). In various embodiments, the system executing the process 200 may utilize distributed processing, parallel processing, cloud processing, and/or edge computing techniques. The process 200 is described below as being executed by the digital therapeutic system 100; accordingly, it should be understood that the functions can be individually or collectively executed by one or multiple devices or subsystems associated with the digital therapeutic system 100.

In particular, the digital therapeutic system 100 executing the process 200 may receive 202 audio and/or video recorded of the user (by themselves or by a third party such as a family member). For example, the received 202 audio and/or video may include video journal entries that the user was prompted to create and upload via the digital therapy app 122, which can include both audio and video content. In one embodiment, the digital therapy app 122 may prompt a user to upload a video and/or audio of themselves describing how they are feeling, either independently or in connection with the provision of the digital therapy content 106 via the digital therapy app 122.

Further, the digital therapeutic system 100 may analyze 204, 206 the uploaded video and/or audio content (via, e.g., the speech analysis module 110) for one or more biomarkers associated with the user. In one embodiment, the digital therapeutic system 100 may analyze 204 only the audio data for audio-based biomarkers. In another embodiment, the digital therapeutic system 100 may analyze 206 only the video data for visual-based biomarkers. In yet another embodiment, the digital therapeutic system 100 may analyze 204, 206 the audio data and the video data in combination with each other for a variety of different biomarkers.

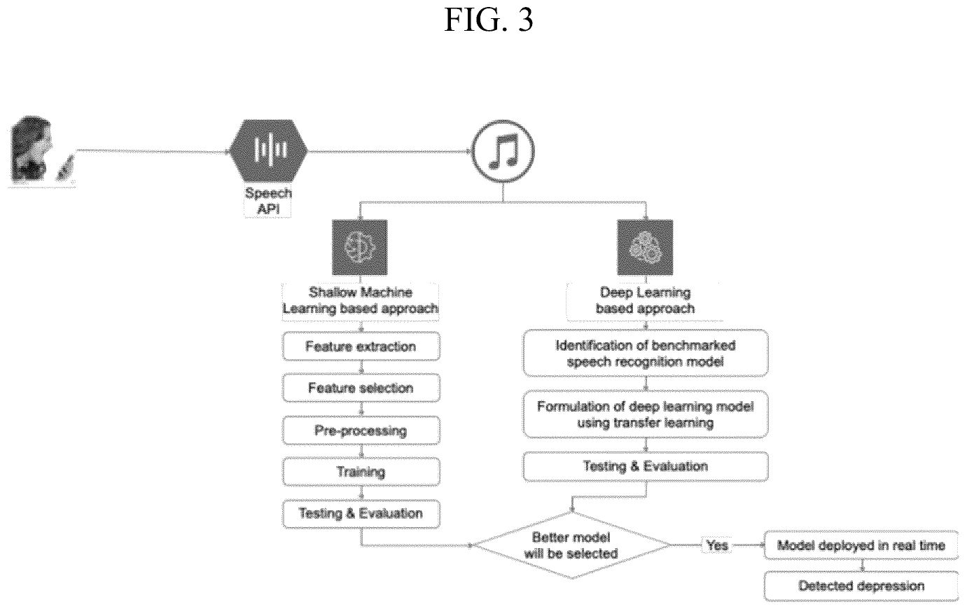

In various embodiments, the digital therapeutic system 100 may analyze 204 the audio data for speech biomarkers associated with the user using a variety of different machine learning (ML)-based and/or algorithmic techniques. The audio-based biomarkers may include a vocal change exhibited by the user (e.g., due to increased muscle tension due to stress or anxiety), speech content, and so on. In one embodiment, the digital therapeutic system 100 may store and execute a ML model trained to for feature extraction and classification of the digital signal processing of speech data. In one embodiment, the digital therapeutic system 100 may analyze 206 the speech content using natural language processing techniques to identify particular words uttered by the user. In some embodiments, the digital therapeutic system 100 may analyze 204, 206 the signal and content of the user's speech using techniques described in U.S. patent application Ser. No. 17/725,145, which is incorporated by reference herein. As shown in FIG. 3, the digital therapeutic system 100 may use shallow ML-based approaches, deep ML-based approaches, or a combination thereof to analyze the user's speech signal and/or speech content.

In one embodiment, the speech analysis module 110 may implement or otherwise include an audio classification model to analyze 204 the audio data. In this embodiment, the audio classification model may be trained or programmed to identify distress conditions (e.g., anxiety, depression, or post-traumatic stress disorder) within an audio sample. As generally described above, users are encouraged to record audio and/or video of themselves as part of a journaling or self-assessment function by the digital therapy app 122. Accordingly, the digital therapeutic system 100, via the speech analysis module 110, may be configured to analyze the audio recorded from a user to identify the presence of such stress conditions. If the user is determined to be exhibiting signs or symptoms of a stress condition, the digital therapeutic system 100 may take a variety of different actions, including adjusting the distal therapy content 106 provided to the user via the digital therapy app 122 or notifying a healthcare provider 140.

In one implementation, an audio classification model executable by the speech analysis module 110 to analyze 204 audio data was built using a residual neural network. A residual neural network is a neural network that has skip connections that connect activations of a layer to further layers by skipping some layers in between. The skip connections form a residual block and the residual neural network is made by stacking residual blocks together. In this implementation, raw audio files were converted into spectrograms, which are then used as inputs for the residual network. The raw audio data may be chunked prior to being converted into spectrograms. In one illustrative implementation, the audio data may be separated into windows of a defined length, a Fast Fourier Transform may be computed for each window to transform the data from time domain to the frequency domain, a Mel scale may be generated to separate the frequency spectrum from the audio data into a defined number of evenly spaced frequencies, and a spectrogram may be calculated for each window corresponding to the frequencies in the Mel scale. The spectrogram for each window was then utilized as input to train the residual network.

In one illustrative embodiment where is input data was not chunked, a sample size of 189 audio files was used, which was split into a training data set of 101 files and a validation data set of 88 files. In another illustrative embodiment where the input data was chunked, a sample size of 5,757 audio files was used, which was split into a training data set of 3,054 files and a validation data set of 2,703 files. In both cases, a residual neural network was trained on the training data set and then validated on the validation data set, as performed in the machine learning technical field. In various implementation, the trained residual network exhibited a 66-75% accuracy on the validation data set. Accordingly, the trained audio classification model was determined to be able to accurately and consistently identify whether a user was exhibiting a stress condition or distress based on audio that users have recorded of themselves.

In various embodiments, the digital therapeutic system 100 may analyze 206 the video data for speech biomarkers associated with the user using a variety of different ML-based and/or algorithmic techniques. The video-based biomarkers may include heart rate (e.g., beats per minute) or heart rate variability (HRV). Determining heart rate or HRV can be useful because such biomarkers are associated with stress and, thus, are useful for identifying whether a user is suffering from distress or anxiety (e.g., due to PPD). In one embodiment, the digital therapeutic system 100 may analyze 206 the video data for visual biomarkers associated with the user using remote photoplethysmography (rPPG) techniques. In the illustrative embodiment shown in FIG. 2, the digital therapeutic system 100 may extract 208 the user's face from the received user video content. In one particular implementation, the user's face may be extracted 208 on a frame-by-frame basis. Accordingly, the digital therapeutic system 100 may identify regions of interest (ROIs) on the extracted 208 images of the user's face. In one embodiment, the ROIs may be classified as skin or non-skin portions of the user's face. For the identified skin ROIs, the digital therapeutic system 100 may determine the user's heart rate using rPPG. In particular, the digital therapeutic system 100 may use RGB-based statistical analysis on the identified skin cells of the corresponding ROIs, which can in turn be used to calculate the user's heartbeat spectrum on a frame-by-frame basis. However, in other embodiments, the digital therapeutic system 100 may utilize other ML-based and/or algorithmic techniques for identifying biomarkers associated with the user.

Based on the audio-based biomarkers and/or video-based biomarkers (e.g., the user's heartbeat), the digital therapeutic system 100 may determine 216 the user's mental state based on demographic and clinically validated scales, such as the Edinburgh Postnatal Depression Scale (EPDS). In some embodiments, the aforementioned functions may be repeated for one or more iterations (e.g., 3-5 days) to develop baseline scores for the user. Accordingly, the digital therapeutic system 100 may track variations in the parameters calculated from the audio and/or video content uploaded by the user. Based on the variations in the calculated parameters over time, the digital therapeutic system 100 may predict the user's state of mind.

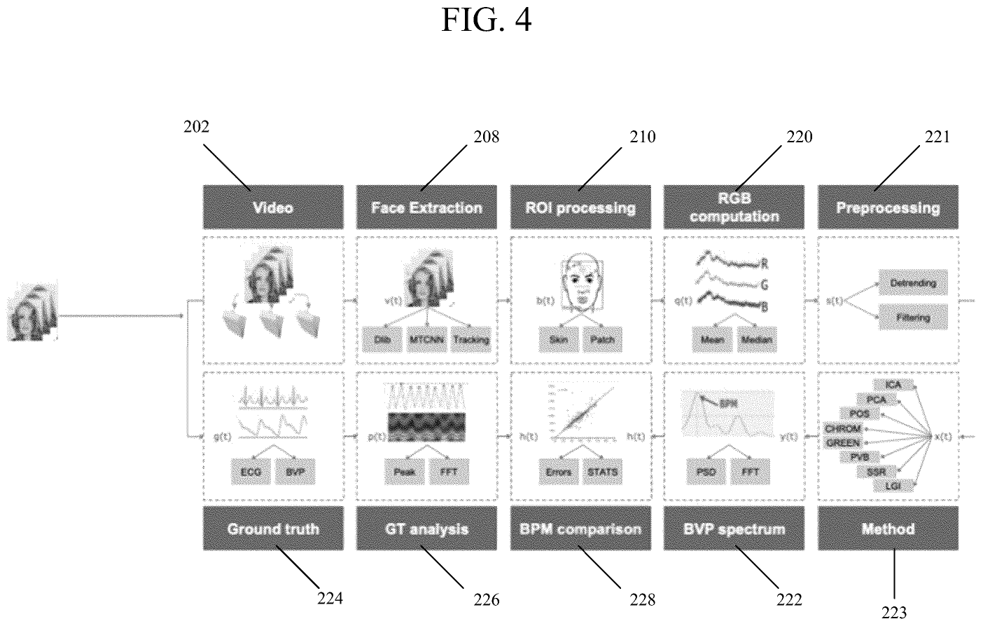

Referring now to FIG. 4, one particular implementation of rPPG techniques to user video data over time is shown. As described above, the digital therapeutic system receives 202 video content uploaded by the user, extracts 208 the user's face from each video frame, and processes the extracted images to identify 210 the ROIs. Once the ROIs have been identified, the digital therapeutic system 100 may compute 220 the RGB values for the skin ROIs on a frame-by-frame basis and determine 222 the blood volume pulse (BVP) spectrum therefrom, which in turn can be used to determine the user's heart rate across the frames of the video content. In some embodiments, the extracted 208 face data may undergo preprocessing 221 prior to calculating 222 the BVP spectrum. In particular, the preprocessing 221 may include de-trending and filtering. In some embodiments, the BVP spectrum may be calculated 222 using a variety of different methods 223, including independent component analysis (ICA), principal components analysis (PCA), point of sale (POS), single-scale retinex (SSR), local gyrification index (LGI), GREEN, CHROM, local group invariance (LGI), and other techniques. Further, the digital therapeutic system 100 may retrieve 224 the ground truth or baseline values previously determined for the user (e.g., from the database 112) and analyze 226 the pre-characterized baseline heart rate values to compare 228 the user's heart rate (i.e., beats per minute) for the particular instance of the uploaded video content to the user's baseline values. If the user's heart rate for the particular uploaded video content deviates by at least a threshold from the user's baseline values, that could indicate that there is an issue with the user's state of mind.

As described above, the digital therapeutic system 100 may implement one or more machine learning models and/or algorithms to execute the functions of the process 200 described above, including analyzing 204 audio data and analyzing 206 video data. In various embodiments, the machine learning models and/or algorithms may include neural networks, decisions trees (e.g., random forests), support vector machines, regressions, hidden Markov models, and other types of machine learning techniques known in the field. Further, the neural networks may include any general category of neural network, including deep neural networks, convolutional neural networks, autoencoders, recurrent neural networks, and so on. Further, the machine learning models described herein may be trained using supervised or unsupervised learning techniques.

In some embodiments, the process 200 executed by the digital therapeutic system 100 may be executed as audio and/or video content is uploaded by the user. Accordingly, the digital therapeutic system 100 may provide 218 the user's predicted state of mind. In various embodiments, the user's predicted state of mind may be provided 218 to the user (e.g., as a push notification or via the UI of the digital therapy app 122) or a healthcare provider 140 (e.g., as an email or a message delivered through a healthcare provider web portal for the digital therapeutic system 100).

It should further be noted that although the functions and/or steps of the process 200 are depicted in a particular order or arrangement, the depicted order and/or arrangement of steps and/or functions is simply provided for illustrative purposes. Unless explicitly described herein to the contrary, the various steps and/or functions of the process 200 can be performed in different orders, in parallel with each other, in an interleaved manner, and so on.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art, all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

The functions and process steps herein may be performed automatically or wholly or partially in response to user command. An activity (including a step) performed automatically is performed in response to one or more executable instructions or device operation without user direct initiation of the activity.

1. A computer system for providing digital therapy content to a user via a user device, the user device comprising a camera and a microphone for recording audio and video of the user, the computer system comprising:
a processor; and
a memory coupled to the processor, the memory storing instructions that, when executed by the processor, cause the computer system to:
provide the digital therapy content to the user device,
receive the audio and the video of the user from the user device in connection with the digital therapy content,
determine a speech-based biomarker associated with the user from the audio,
determine a visual-based biomarker associated with the user from the video via remote photoplethysmography,
determine a mental state for the user based on at least one of the determined speech-based biomarker or the determined visual-based biomarker of the user, and
provide the determined mental state to at least one of the user or a healthcare provider associated with the user.

2. The computer system of claim 1, wherein the determined mental state is provided via a user interface of a digital therapy app executed by the user device, wherein the digital therapy app is communicably coupled to the computer system.

3. The computer system of claim 1, wherein the digital therapy content is configured for treatment of postpartum depression.

4. The computer system of claim 1, wherein the memory further stores a machine learning model trained to identify a distress condition based on audio input data, wherein the speech marker is determined based on the machine learning model.

5. The computer system of claim 1, wherein the mental state is determined based on both the determined speech biomarker and the determined visual biomarker.

6. The computer system of claim 1, wherein the mental state comprises at least one of anxiety, depression, or post-traumatic stress disorder.

7. The computer system of claim 1, wherein the memory stores further instructions that, when executed by the processor, adjust the digital therapy content provided to the user based on the determined state of mind.

8. A computer-implemented method for providing digital therapy content to a user via a user device, the user device comprising a camera and a microphone for recording audio and video of the user, the method comprising:
providing, by a computer system, the digital therapy content to the user device;
receiving, by the computer system, the audio and the video of the user from the user device in connection with the digital therapy content;
determining, by the computer system, a speech-based biomarker associated with the user from the audio;
determining, by the computer system, a visual-based biomarker associated with the user from the video via remote photoplethysmography;
determining, by the computer system, a mental state for the user based on at least one of the determined speech-based biomarker or the determined visual-based biomarker of the user; and
providing, by the computer system, the determined mental state to at least one of the user or a healthcare provider associated with the user.

9. The method of claim 8, wherein the determined mental state is provided via a user interface of a digital therapy app executed by the user device, wherein the digital therapy app is communicably coupled to the computer system.

10. The method of claim 8, wherein the digital therapy content is configured for treatment of postpartum depression.

11. The method of claim 8, wherein the computer system executes a machine learning model trained to identify a distress condition based on audio input data, wherein the speech marker is determined based on the machine learning model.

12. The method of claim 8, wherein the mental state is determined based on both the determined speech biomarker and the determined visual biomarker.

13. The method of claim 8, wherein the mental state comprises at least one of anxiety, depression, or post-traumatic stress disorder.

14. The method of claim 8, further comprising:
adjusting, by the computer system, the digital therapy content provided to the user based on the determined state of mind.
